(19) United States
(12) Patent Application Publication
(10) Pub. No.: US 2026/0232960 A1
(43) Pub. Date: Aug. 13, 2026

(54) STEERABLE CATHETER

(71) Applicant: BOSTON SCIENTIFIC SCIMED, INC., MAPLE GROVE, MN (US)

(72) Inventors: Sarah Tian-Hong Hua, Plymouth, MN (US); Carly Rogers, Eden Prairie, MN (US); Vail Baumer, Minneapolis, MN (US)

(73) Assignee: BOSTON SCIENTIFIC SCIMED, INC., MAPLE GROVE, MN (US)

(21) Appl. No.: 19/532,316

(22) Filed: Feb. 6, 2026

Related U.S. Application Data
(60) Provisional application No. 63/755,522, filed on Feb. 7, 2025.

(51) Int. Cl. A61M 25/01

(52) U.S. Cl. CPC A61M 25/0136; A61M 25/0147; A61M 2025/015; A61M 2205/582

(57) ABSTRACT

The present technology relates to steerable catheters that can include an indicator assembly for providing an indication to a user of a direction and/or magnitude of steering of the catheter shaft including various components of the indicator assembly and handle of the catheter shaft. In certain examples, steerable catheters may include a tensioning mechanical for pull-wires and pressure sensors.

### CROSS-REFERENCE TO RELATED APPLICATIONS

This application claims the benefit of priority of U.S. Provisional Application No. 63/755,522 filed Feb. 7, 2025, the entire disclosure of which is hereby incorporated by reference.

### TECHNICAL FIELD

The disclosure relates generally to medical devices and more particularly to steerable catheters, sheaths, and/or elongate tubular shafts.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

In one example, a steerable catheter may comprise a handle assembly comprising a handle shell and a steering mechanism, and an elongate sheath extending distally from the handle assembly. The steering mechanism may include an axial translation mechanism coupled to a first steering wire extending within the elongate sheath and a second steering wire extending within the elongate sheath. The first steering wire may be configured to cooperate with the axial translation mechanism to bend a distal portion of the elongate sheath in a first direction. The second steering wire may be configured to cooperate with the axial translation mechanism to bend the distal portion of the elongate sheath in a second direction opposite the first direction. The handle assembly may comprise a non-visual torque indicator configured to convey a rotational position of the handle assembly relative to a neutral position.

In addition, or alternatively, to any example disclosed herein, the non-visual torque indicator comprises a tactile feature formed on a hand grip portion of the handle shell.

In addition, or alternatively, to any example disclosed herein, the tactile feature comprises a bumped-out region of the handle shell extending longitudinally along the handle shell.

In addition, or alternatively, to any example disclosed herein, the bumped-out region is disposed between a proximal end of the hand grip portion and a distal end of the hand grip portion.

In addition, or alternatively, to any example disclosed herein, the tactile feature extends along more than 50% of a length of the hand grip portion.

In addition, or alternatively, to any example disclosed herein, the tactile feature extends along less than 100% of the length of the hand grip portion.

In addition, or alternatively, to any example disclosed herein, the hand grip portion comprises a plurality of grooves configured to receive a user's fingers therein.

In addition, or alternatively, to any example disclosed herein, the axial translation mechanism comprises an elongate shaft having a threaded portion disposed within the handle shell, a first carriage member operatively engaged with the threaded portion, and a second carriage member operatively engaged with the threaded portion. Rotation of the elongate shaft is configured to translate the first carriage member and the second carriage member in opposite directions along the elongate shaft.

In addition, or alternatively, to any example disclosed herein, the handle assembly comprises a knob fixedly attached to the elongate shaft outside of the handle shell, wherein the knob is rotatable relative to the handle shell to rotate the threaded portion within the handle shell.

In addition, or alternatively, to any example disclosed herein, the knob is axially offset from the handle shell.

In addition, or alternatively, to any example disclosed herein, and in a second example, a steerable catheter may comprise a handle assembly comprising a handle shell and a steering mechanism, and an elongate sheath extending distally from the handle assembly. The steering mechanism may include an axial translation mechanism. A first steering wire may extend distally from the axial translation mechanism to a distal pull ring disposed within the elongate sheath. A second steering wire may extend distally from the axial translation mechanism to the distal pull ring, the second steering wire being disposed on an opposite side of the elongate sheath from the first steering wire relative to a central longitudinal axis of the elongate sheath. The first steering wire may be configured to cooperate with the axial translation mechanism to bend a distal portion of the elongate sheath in a first direction. The second steering wire may be configured to cooperate with the axial translation mechanism to bend the distal portion of the elongate sheath in a second direction opposite the first direction. The handle assembly may comprise a non-visual torque indicator configured to convey a rotational position of the handle assembly relative to a neutral position.

In addition, or alternatively, to any example disclosed herein, the axial translation mechanism is configured to shift the elongate sheath between a relaxed configuration, a deflected configuration wherein the distal portion of the elongate sheath is bent in the first direction from the relaxed configuration, and a straightened configuration wherein the distal portion of the elongate sheath is bent in the second direction from the relaxed configuration.

In addition, or alternatively, to any example disclosed herein, actuation of the axial translation mechanism is configured to selectively apply tension to the first steering wire and the second steering wire to shift the elongate sheath between the relaxed configuration, the deflected configuration, and the straightened configuration.

In addition, or alternatively, to any example disclosed herein, the axial translation mechanism comprises an elongate shaft having a threaded portion disposed within the handle shell, a first carriage member operatively engaged with the threaded portion, and a second carriage member operatively engaged with the threaded portion. Rotation of the elongate shaft is configured to translate the first carriage member and the second carriage member in opposite directions along the elongate shaft.

In addition, or alternatively, to any example disclosed herein, the handle assembly comprises a knob fixedly attached to the elongate shaft outside of the handle shell, wherein the knob is rotatable relative to the handle shell to rotate the threaded portion within the handle shell.

In addition, or alternatively, to any example disclosed herein, the knob is proximally offset from the handle shell.

In addition, or alternatively, to any example disclosed herein, the knob is distally offset from the handle shell.

In addition, or alternatively, to any example disclosed herein, the threaded portion comprises a first helical thread extending in a first helical direction overlapping a second helical thread extending in a second helical direction.

In addition, or alternatively, to any example disclosed herein, the first carriage member is coupled to the first steering wire and the second carriage member is coupled to the second steering wire.

In addition, or alternatively, to any example disclosed herein, and in a third example, a steerable catheter may comprise a handle assembly comprising a handle shell and a steering mechanism, and an elongate sheath extending distally from the handle assembly. The steering mechanism may include an axial translation mechanism. A first steering wire may extend distally from the axial translation mechanism to a distal pull ring disposed within the elongate sheath. A second steering wire may extend distally from the axial translation mechanism to the distal pull ring, the second steering wire being disposed on an opposite side of the elongate sheath from the first steering wire relative to a central longitudinal axis of the elongate sheath. The first steering wire may be configured to cooperate with the axial translation mechanism to bend a distal portion of the elongate sheath in a first direction. The second steering wire may be configured to cooperate with the axial translation mechanism to bend the distal portion of the elongate sheath in a second direction opposite the first direction. The handle assembly may comprise a non-visual torque indicator configured to convey a rotational position of the handle assembly relative to a neutral position. The handle assembly may be configured to permit ambidextrous operation of the steering mechanism.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of an example steerable catheter;

FIG. 2 illustrates selected aspects of the example steerable catheter;

FIG. 3 illustrates selected aspects of the example steerable catheter;

FIG. 3A is a cross-sectional view of FIG. 3 taken along line 3A-3A;

FIG. 4 illustrates selected aspects of an alternative configuration of the example steerable catheter;

FIG. 5 illustrates selected aspects of an alternative configuration of the example steerable catheter;

FIG. 6 illustrates selected aspects of an alternative configuration of the example steerable catheter;

FIG. 7 illustrates selected aspects of an alternative configuration of the example steerable catheter of FIG. 6;

FIG. 8 illustrates selected aspects of an alternative configuration of the example steerable catheter;

FIG. 9 illustrates selected aspects of an alternative configuration of the example steerable catheter; and

FIG. 10 illustrates selected aspects of an alternative configuration of the example steerable catheter.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

In some medical procedures, delivery and/or access sheaths may be routed percutaneously into a body cavity, lumen, and/or treatment site. Navigation through patient vasculature and/or organs may include steering through tortuous anatomy and/or directing a distal end of the delivery and/or access sheath into a body cavity, lumen, and/or treatment site. Examples of medical devices suitable for use in medical procedures, such as but not limited to left atrial appendage closure, aortic valve replacement, mitral valve replacement, septal defect repair, etc., are described herein. Existing medical devices may have certain advantages and/or disadvantages. There is an ongoing need for alternative steerable medical devices for delivering medical implants and/or conducting other treatment procedures.

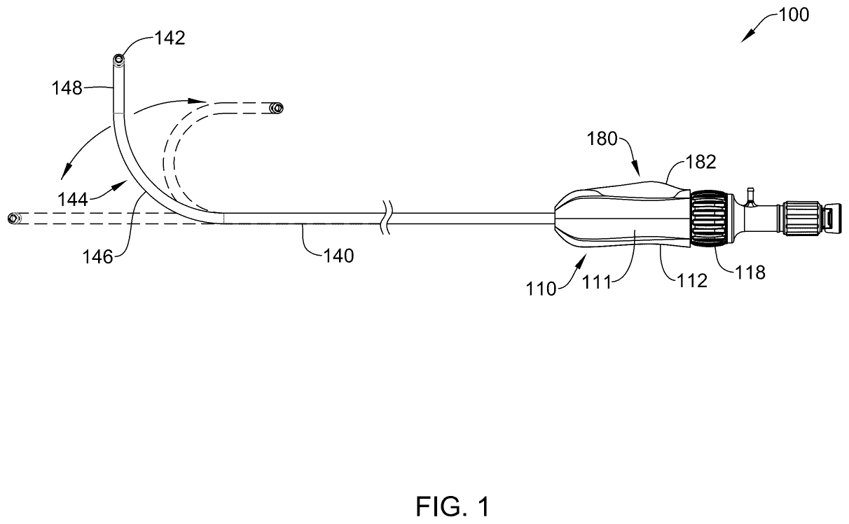

FIG. 1 illustrates selected aspects of a steerable catheter 100. In some embodiments, the steerable catheter 100 may be any one of a variety of catheters, such as an intravascular catheter. Examples of intravascular catheters may include, but are not limited to, balloon catheters, atherectomy catheters, device delivery catheters, drug delivery catheters, diagnostic catheters, and guide catheters. In some embodiments, the steerable catheter 100 may take the form of other suitable guiding, diagnosing, or treating devices (including endoscopic instruments, laparoscopic instruments, etc., and the like) and it may be suitable for use at various locations and/or body lumens within a patient. While the steerable catheter 100 is illustrated and described in one example configuration as a bi-directional steerable catheter, unidirectional, onmidirectional, and/or other types of steerable catheters may also benefit from aspects of the disclosure and therefore the steerable catheter 100 shall not be limited to a bi-directional configuration unless explicitly called out or recited as such.

The steerable catheter 100 may comprise a handle assembly 110 and an elongate sheath 140 extending distally from the handle assembly 110. In some embodiments, the steerable catheter 100 and/or the handle assembly 110 may include a guidewire port, a side port, a fluid flush port, an imaging access port, a Luer connector, and/or other suitable ports, access points, or functional features.

In some embodiments, the elongate sheath 140 may extend into and/or through the handle assembly 110. The handle assembly 110 may comprise a handle housing. In some embodiments, the elongate sheath 140 may extend into and/or through a distal opening in the handle housing. In some embodiments, the elongate sheath 140 may be axially secured relative to the handle housing. In some embodiments, a proximal portion of the elongate sheath 140 may include a key element configured to non-rotatably engage one or more lock elements fixedly attached to an inner surface of the handle housing. In some embodiments, the key element may be bonded to an outer surface of the elongate sheath 140. In some embodiments, the key element may be monolithically formed with the elongate sheath 140. In some embodiments, the key element may be welded (e.g., heat weld, sonic weld, vibration weld, etc.) to the elongate sheath 140. In some embodiments, the key element may be melted together (e.g., reflowed) with the elongate sheath 140 such that material of the key element is co-mingled with material of the elongate sheath 140 at a molecular level. In some embodiments, the handle housing may include one or more lock elements fixedly attached to and/or monolithically formed with the inner surface of the handle housing. In some embodiments, the one or more lock elements may be formed as ribs or other structural support members configured to increase the rigidity of the handle housing and permit torque transfer between the handle housing and the elongate sheath 140. In some embodiments, the elongate sheath 140 may have a relaxed configuration. The elongate sheath 140 is shown in the relaxed configuration in solid lines in FIG. 1. The elongate sheath 140 may be self-biased toward, and/or in the absence of any outside forces may return to, the relaxed configuration.

In some embodiments, the elongate sheath 140 may include an atraumatic distal tip 142. In some embodiments, the elongate sheath 140 may include a distal portion 144 having a first curve 146 and a second curve 148, such that the elongate sheath 140 has a preset double curve, in the relaxed configuration, as shown in FIG. 1. In some embodiments, the first curve 146 may be preset to curve upwards, as viewed in FIG. 1. Other configurations are also contemplated. In some embodiments, the second curve 148 may be preset to curve to the left, as viewed proximally to distally along the elongate sheath 140. Other configurations are also contemplated.

In some embodiments, the distal portion 144 and/or the first curve 146 may be configured to bend or deflect in a first direction, wherein the atraumatic distal tip 142 is bent and/or moved towards and/or closer to the handle assembly 110, toward and/or to a deflected configuration, as shown in phantom in FIG. 1. In some embodiments, the distal portion 144 and/or the first curve 146 may be configured to bend or deflect in a second direction opposite the first direction, wherein the atraumatic distal tip 142 is bent and/or moved away from and/or farther from the handle assembly 110, toward and/or to a straightened configuration, as shown in phantom in FIG. 1. In some embodiments, the elongate sheath 140 may have only a single curve in the normal or relaxed configuration. In some embodiments, the elongate sheath 140 may be substantially straight in the normal or relaxed configuration. Other configurations, including combinations of those described herein, are also contemplated.

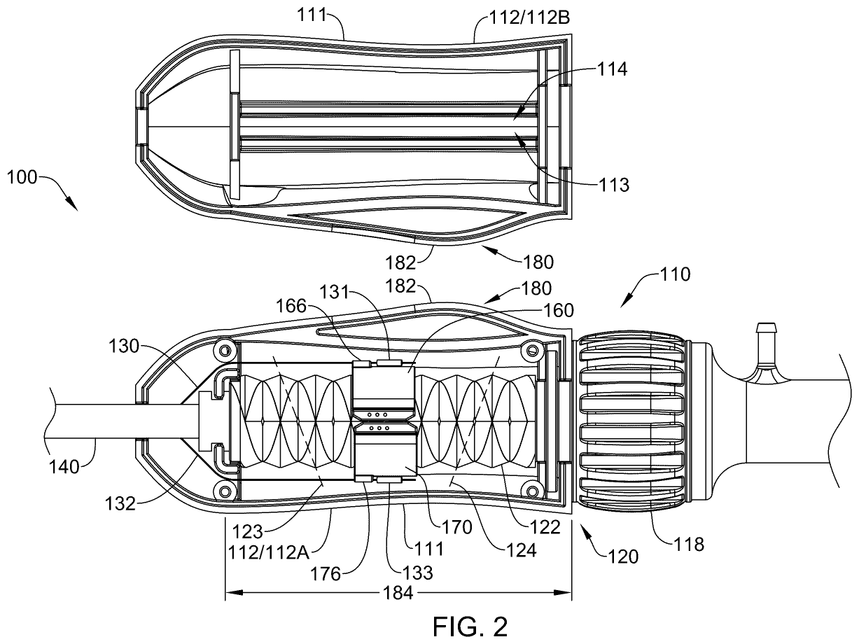

FIG. 2 illustrates selected features of the steerable catheter 100 and the handle assembly 110. The handle assembly 110 and/or the handle housing may comprise a handle shell 112. In some embodiments, the handle shell 112 may comprise a first handle shell 112A (e.g., a right handle shell, a bottom handle shell, etc.) and a second handle shell 112B (e.g., a left handle shell, a top handle shell, etc.). In some embodiments, the handle shell 112 may form a clam shell configuration. In some embodiments, the first handle shell 112A and the second handle shell 112B may be secured together using fasteners, a snap fit, an interference fit, etc. Some suitable but non-limiting materials for the handle assembly 110, the handle housing, the handle shell 112, the first handle shell 112A, the second handle shell 112B, etc., including but not limited to polymeric materials, metallic materials, and/or composite materials, are described below. In some embodiments, the handle assembly 110, the handle housing, the handle shell 112, the first handle shell 112A, the second handle shell 112B may preferably be formed from a polymeric material. In one non-limiting example, the handle assembly 110, the handle housing, the handle shell 112, the first handle shell 112A, the second handle shell 112B may preferably be formed from acrylonitrile butadiene styrene (ABS). Other configurations and/or materials are also contemplated. In the view shown in FIG. 2, the second handle shell 112B has been flipped over to show some internal components of the handle assembly 110.

In some embodiments, the handle assembly 110 may comprise a steering mechanism. In some embodiments, the steering mechanism may include an axial translation mechanism 120 disposable within and/or disposed within the handle assembly 110 and/or the handle shell 112 of the steerable catheter 100. In some embodiments, the axial translation mechanism 120 may comprise an elongate shaft 122 having a circumference. In some embodiments, the elongate shaft 122 may have a threaded portion disposed within the handle housing and/or the handle shell 112. In some embodiments, the elongate shaft 122 may be rotatably disposed within the handle housing and/or the handle shell 112. In some embodiments, the elongate shaft 122 may be rotatable within and/or relative to the handle housing and/or the handle shell 112.

In some embodiments, the elongate shaft 122 and/or the threaded portion may comprise a first helical thread 123 extending along a length of the elongate shaft 122 in a first helical direction. In some embodiments, the elongate shaft 122 and/or the threaded portion may comprise a second helical thread 124 extending along the length of the elongate shaft 122 in a second helical direction different from the first helical direction. In some embodiments, the first helical thread 123 may extend along an exterior of the elongate shaft 122 and/or the threaded portion in the first helical direction. In some embodiments, the second helical thread 124 may extend along the exterior of the elongate shaft 122 and/or the threaded portion in the second helical direction. In some embodiments, the first helical thread 123 may axially overlap the second helical thread 124. In some embodiments, the first helical thread 123 may intersect the second helical thread 124. In some embodiments, the elongate shaft 122 and/or the threaded portion may comprise the first helical thread 123 extending in the first helical direction axially overlapping the second helical thread extending the second helical direction, wherein the second helical direction is different from the first helical direction. In some embodiments, the first helical thread 123 may be discontinuous along the elongate shaft 122 and/or the threaded portion. In some embodiments, the second helical thread 124 may be discontinuous along the elongate shaft 122 and/or the threaded portion.

In some embodiments, the threaded portion of the elongate shaft 122 may comprise clockwise threading (e.g., the first helical thread 123) extending in a proximal to distal direction overlaid with counterclockwise threading (e.g., the second helical thread 124) extending in the proximal to distal direction. In some embodiments, the clockwise threading (e.g., the first helical thread 123) and the counterclockwise threading (e.g., the second helical thread 124) are formed by intersecting helical grooves extending radially inward from an outermost extent of the elongate shaft 122 and/or the threaded portion. In some embodiments, the intersecting helical grooves may form a plurality of diamond-shaped projections extending radially outward from an innermost radial extent of the intersecting helical grooves. In some embodiments, each diamond-shaped projection of the plurality of diamond-shaped projections forms a portion of the clockwise threading (e.g., the first helical thread 123) and a portion of the counterclockwise threading (e.g., the second helical thread 124).

In some embodiments, the elongate sheath 140 may extend within and/or through the elongate shaft 122. In some embodiments, the elongate sheath 140 may be disposed coaxial with the elongate shaft 122. In some embodiments, the elongate shaft 122 may be configured to rotate around and/or relative to the elongate sheath 140.

In some embodiments, the steerable catheter 100 may comprise a knob 118, wherein the knob 118 is rotatable and/or is configured to rotate relative to the handle housing and/or the handle shell 112 to rotating the threaded portion of the elongate shaft 122 within the handle housing and/or the handle shell 112. In some embodiments, the knob 118 may be fixedly attached to the elongate shaft 122 outside of the handle housing and/or the handle shell 112. In some embodiments, the knob 118 may be monolithically formed with the elongate shaft 122. In some embodiments, the knob 118 may be configured to rotate around and/or relative to the elongate sheath 140. In some embodiments, rotation of the knob 118 relative to the handle housing and/or the handle shell 112 causes rotation of the elongate shaft 122 relative to the handle housing and/or the handle shell 112. Some suitable but non-limiting materials for the elongate shaft 122 and/or the knob 118, including but not limited to polymeric materials, metallic materials, and/or composite materials, are described below. In some embodiments, the elongate shaft 122 and/or the knob 118 may preferably be formed from a polymeric material. In one non-limiting example, the elongate shaft 122 and/or the knob 118 may preferably be formed from acrylonitrile butadiene styrene (ABS). Other configurations and/or materials are also contemplated.

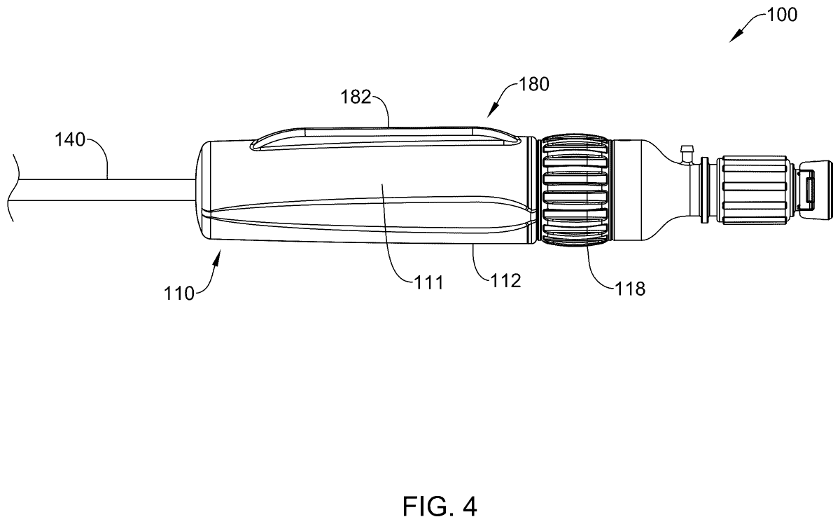

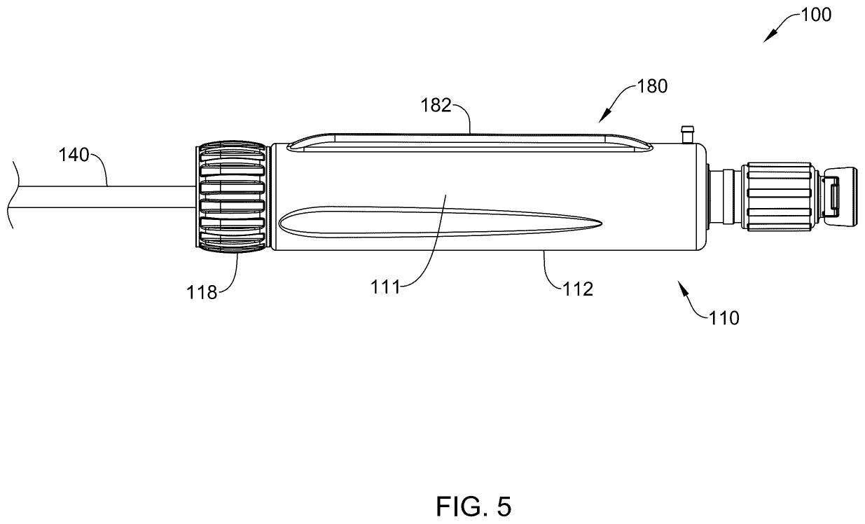

In some embodiments, the knob 118 may be axially offset from the handle housing and/or the handle shell 112. In some embodiments, the knob 118 may be proximally offset from the handle housing and/or the handle shell 112, as seen in FIGS. 1 and 4 for example. In some embodiments, the knob 118 may be distally offset from the handle housing and/or the handle shell 112, as seen in FIG. 5 for example. Other configurations are also contemplated. In some embodiments, the knob 118 may be axially spaced apart from the threaded portion of the elongate shaft 122. In some embodiments, the knob 118 may be axially spaced apart from the first helical thread 123 and the second helical thread 124.

In some embodiments, the steering mechanism may comprise the axial translation mechanism 120 coupled to a first steering wire 130 extending within the elongate sheath 140 and a second steering wire 132 extending within the elongate sheath 140, as seen in FIG. 2. In some embodiments, the first steering wire 130 may be configured to cooperate with the axial translation mechanism 120 to bend the distal portion 144 of the elongate sheath 140 in the first direction. In some embodiments, the second steering wire 132 may be configured to cooperate with the axial translation mechanism 120 to bend the distal portion 144 of the elongate sheath 140 in the second direction. In some embodiments, the steerable catheter 100 may be a bi-directional steerable catheter. Other configurations are also contemplated.

In some embodiments, a first carriage member 160 may be coupled to the first steering wire 130. In some embodiments, the first carriage member 160 may be operatively engaged with the threaded portion of the elongate shaft 122. In some embodiments, the first carriage member 160 may be operatively engaged with the first helical thread 123 and/or the clockwise threading. In some embodiments, a second carriage member 170 may be coupled to the second steering wire 132. In some embodiments, the second carriage member 170 may be operatively engaged with the threaded portion of the elongate shaft 122. In some embodiments, the second carriage member 170 may be operatively engaged with the second helical thread 124 and/or the counterclockwise threading.

In some embodiments, the handle housing and/or the handle shell 112 may comprise one or more slide features configured to guide and/or constrain the first carriage member 160 and the second carriage member 170. In some embodiments, the one or more slide features may permit axial sliding movement of the first carriage member 160 and the second carriage member 170 within the handle housing and/or the handle shell 112. In some embodiments, the one or more slide features may limit and/or constrain the first carriage member 160 and the second carriage member 170 to axial sliding movement within the handle housing and/or the handle shell 112.

In some embodiments, the first handle shell 112A may comprise a first guide slot (not shown) extending along an inner surface of the first handle shell 112A and configured to engage with and/or receive the first carriage member 160, and a second guide slot (not shown) extending along the inner surface of the first handle shell 112A and configured to engage with and/or receive the second carriage member 170. In some embodiments, the second handle shell 112B may comprise a first guide slot 113 extending along an inner surface of the second handle shell 112B and configured to engage with and/or receive the first carriage member 160, and a second guide slot 114 extending along the inner surface of the second handle shell 112B and configured to engage with and/or receive the second carriage member 170. The first guide slot 113 of the second handle shell 112B may be disposed opposite the first guide slot of the first handle shell 112A with respect to a plane containing a central longitudinal axis of the handle assembly 110 and/or a central longitudinal axis of the elongate shaft 122, and the second guide slot 114 of the second handle shell 112B may be disposed opposite the second guide slot of the first handle shell 112A with respect to the plane containing the central longitudinal axis of the handle assembly 110 and/or the central longitudinal axis of the elongate shaft 122. In some embodiments, the first guide slot and the second guide slot of the first handle shell 112A may be spaced apart from each other. In some embodiments, the first guide slot and the second guide slot of the first handle shell 112A may be formed as a single guide slot. In some embodiments, the first guide slot 113 and the second guide slot 114 of the second handle shell 112B may be spaced apart from each other. In some embodiments, the first guide slot 113 and the second guide slot 114 of the second handle shell 112B may be formed as a single guide slot. Other configurations are also contemplated.

In some embodiments, the first carriage member 160 may comprise a first slide extension and a second slide extension opposite the first slide extension. The first slide extension of the first carriage member 160 may be configured to slidably engage and/or may be slidably engaged with the first guide slot of the first handle shell 112A. The second slide extension of the first carriage member 160 may be configured to slidably engage and/or may be slidably engaged with the first guide slot 113 of the second handle shell 112B. In some embodiments, the first carriage member 160 may extend circumferentially around at least a portion of the circumference of the elongate shaft 122. In some embodiments, the first carriage member 160 may extend circumferentially around less than half of the circumference of the elongate shaft 122. In some embodiments, the first carriage member 160 may comprise a concave shape facing toward the elongate shaft 122. In some embodiments, the first carriage member 160 may comprise a first ear 166 extending radially outward from the elongate shaft 122 and/or extending away from the concave shape of the first carriage member 160 facing toward the elongate shaft 122. The first ear 166 may be configured to receive and/or engage with the first steering wire 130. In some embodiments, the first steering wire 130 may pass through an aperture formed in the first ear 166. In some embodiments, the first steering wire 130 may comprise a first wire lug 131 disposed proximal of the first ear 166, wherein the first wire lug 131 is configured to engage with the first ear 166 and is unable to pass through the aperture formed in the first ear 166. In some embodiments, the first wire lug 131 may be configured to couple the first steering wire 130 to the first carriage member 160 and/or the first ear 166.

In some embodiments, the second carriage member 170 may comprise a first slide extension and a second slide extension. The first slide extension of the second carriage member 170 may be configured to slidably engage and/or may be slidably engaged with the second guide slot of the first handle shell 112A. The second slide extension of the second carriage member 170 may be configured to slidably engage and/or may be slidably engaged with the second guide slot 114 of the second handle shell 112B. In some embodiments, the second carriage member 170 may extend circumferentially around at least a portion of the circumference of the elongate shaft 122. In some embodiments, the second carriage member 170 may extend circumferentially around less than half of the circumference of the elongate shaft 122. In some embodiments, the second carriage member 170 may comprise a concave shape facing toward the elongate shaft 122. In some embodiments, the second carriage member 170 may comprise a second ear 176 extending radially outward from the elongate shaft 122 and/or extending away from the concave shape of the second carriage member 170 facing toward the elongate shaft 122. The second ear 176 may be configured to receive and/or engage with the second steering wire 132. In some embodiments, the second steering wire 132 may pass through an aperture formed in the second ear 176. In some embodiments, the second steering wire 132 may comprise a second wire lug 133 disposed proximal of the second ear 176, wherein the second wire lug 133 is configured to engage with the second ear 176 and is unable to pass through the aperture formed in the second ear 176. In some embodiments, the second wire lug 133 may be configured to couple the second steering wire 132 to the second carriage member 170 and/or the second ear 176.

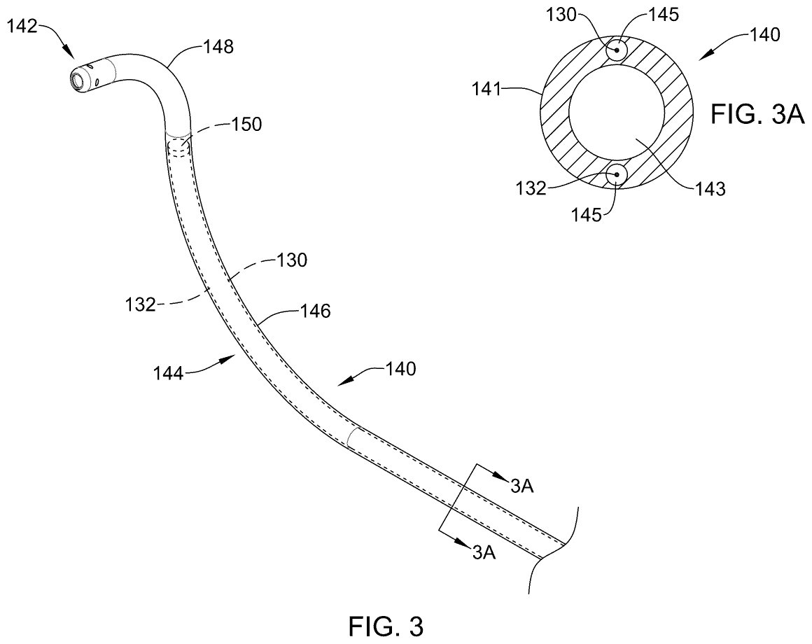

Turning now to FIG. 3, which illustrates selected aspects of the example steerable catheter, and FIG. 3A, which is a cross-sectional view of FIG. 3 taken along the line 3A-3A, the elongate sheath 140 may include a wall 141 defining a central lumen 143 extending from a proximal end to the soft and/or atraumatic distal tip 142 along the central longitudinal axis of the elongate sheath 140. In some embodiments, the central lumen 143 may be coaxial with the central longitudinal axis of the elongate sheath 140. In some embodiments, the central lumen 143 may be a guidewire lumen. In some embodiments, the central lumen 143 may be a device lumen used to deliver a medical device or implant. In some embodiments, the central lumen 143 may have multiple uses. The elongate sheath 140 may include a plurality of steering wire lumens 145 extending and/or disposed within the wall 141. In some embodiments, the plurality of steering wire lumens 145 may include a first steering wire lumen and a second steering wire lumen. In some embodiments, the plurality of steering wire lumens 145 may include more than two steering wire lumens. In some embodiments, the plurality of steering wire lumens 145 may be oriented substantially parallel to the central lumen 143 and/or the central longitudinal axis of the elongate sheath 140. In some embodiments, the plurality of steering wire lumens 145 may be disposed opposite each other and/or on opposite sides of the elongate sheath 140 relative to the central lumen 143 and/or the central longitudinal axis of the elongate sheath 140. Other configurations are also contemplated.

In some embodiments, the plurality of steering wire lumens 145 may have a circular cross-sectional shape, as shown in FIG. 3A. However, the cross-sectional shape shown in FIG. 3A is merely exemplary and is not intended to be limiting. In some embodiments, the plurality of steering wire lumens 145 may have other cross-sectional shapes. For example, in some embodiments, the plurality of steering wire lumens 145 may have a rectangular cross-sectional shape, an ovoid cross-sectional shape, a square cross-sectional shape, a polygonal cross-sectional shape, etc. In some embodiments, the plurality of steering wire lumens 145 may have a cross-sectional shape that is regular and/or symmetrical. In some embodiments, the plurality of steering wire lumens 145 may have a cross-sectional shape that is irregular and/or asymmetrical. Other configurations are also contemplated.

In some embodiments, a distal pull ring 150 may be disposed within the distal portion 144 of the elongate sheath 140. In some embodiments, the distal pull ring 150 may be disposed proximal to the second curve 148 and/or the atraumatic distal tip 142. In some embodiments, the distal pull ring 150 may be disposed proximate a distal end of the first curve 146. In some embodiments, the distal pull ring 150 may be embedded within the wall 141 of the elongate sheath 140. In some embodiments, the distal pull ring 150 may be secured, bonded, and/or fixedly attached to an inner surface of the wall 141 of the elongate sheath 140. Other configurations are also contemplated. Some suitable but non-limiting materials for the distal pull ring 150 are described below.

The first steering wire 130 and the second steering wire 132 may each be slidably disposed within the plurality of steering wire lumens 145. In one example, the first steering wire 130 may be slidably disposed within a first steering wire lumen and the second steering wire 132 may be disposed within a second steering wire lumen. The first steering wire 130 and the second steering wire 132 may be fixedly attached (e.g., bonded, welded, etc.) to the distal pull ring 150. For example, a distal end of the first steering wire 130 may be fixedly attached to the distal pull ring 150 and a distal end of the second steering wire 132 may be fixedly attached to the distal pull ring 150 at a position opposite the distal end of the first steering wire 130 relative to the central longitudinal axis of the elongate sheath 140. Some suitable but non-limiting materials for the first steering wire 130 and the second steering wire 132 are described below.

In some embodiments, the elongate sheath 140 may be sized in accordance with its intended use. For example, the elongate sheath 140 can have a length that is in the range of about 50 to about 200 centimeters (19.69 to 78.74 inches), about 75 to about 175 centimeters (29.53 to 68.90 inches), or about 100 to about 150 centimeters (39.37 to 59.06 inches). Other lengths are also contemplated. It is further contemplated that the outer diameter of the elongate sheath 140 may vary based on the use or application. In some examples, the outer diameter of the elongate sheath 140 may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm (or 18 French), about 6.5 mm, about 7 mm (or 21 French), about 8 mm (or 24 French), or other suitable sizes. In some embodiments, the outer diameter of the elongate sheath 140 may be a maximum of 5.66 mm (17 French), and is preferably smaller than 5.66 mm (17 French). Other configurations are also contemplated. Some suitable but non-limiting materials for the elongate sheath 140, which may include polymeric materials, metallic materials, composite materials, etc., are described below.

The first steering wire 130 may extend distally from the axial translation mechanism 120 and/or the first carriage member 160 to the distal pull ring 150 disposed within the elongate sheath 140. The second steering wire 132 may extend distally from the axial translation mechanism 120 and/or the second carriage member 170 to the distal pull ring 150 disposed within the elongate sheath 140. The first steering wire 130 may extend distally within the elongate sheath 140 (e.g., within the first steering wire lumen of the plurality of steering wire lumens 145) from the handle housing and/or within the handle shell 112 to the distal pull ring 150. The second steering wire 132 may extend distally within the elongate sheath 140 (e.g., within the second steering wire lumen of the plurality of steering wire lumens 145) from the handle housing and/or the handle shell 112 to the distal pull ring 150. The second steering wire 132 may be disposed on an opposite side of the elongate sheath 140 from the first steering wire 130 relative to a central longitudinal axis of the elongate sheath 140.

In some embodiments, the steering mechanism and/or the axial translation mechanism 120 may be configured to shift the elongate sheath 140 extending distally from the handle assembly 110 between the relaxed configuration, the deflected configuration, and the straightened configuration.

In some embodiments, rotation of the knob 118 in a clockwise direction, as viewed along the steerable catheter 100 in the proximal to distal direction, may cause clockwise rotation of the elongate shaft 122 within and/or relative to the handle housing and/or the handle shell 112. In some embodiments, rotation of the knob 118 in a counterclockwise direction, as viewed along the steerable catheter 100 in the proximal to distal direction, may cause counterclockwise rotation of the elongate shaft 122 within and/or relative to the handle housing and/or the handle shell 112.

In some embodiments, rotation of the knob 118 and/or the elongate shaft 122 may be configured to actuate the axial translation mechanism 120. In some embodiments, rotation of the knob 118 and/or the elongate shaft 122 may be configured to axially translate the first carriage member 160 and the second carriage member 170 along the threaded portion of the elongate shaft 122 and/or within the handle housing and/or the handle shell 112. In some embodiments, rotation of the knob 118 and/or the elongate shaft 122 may be configured to axially translate the first carriage member 160 and the second carriage member 170 in opposite directions along the threaded portion of the elongate shaft 122 and/or within the handle housing and/or the handle shell 112. In some embodiments, actuation of the axial translation mechanism 120 may be configured to selectively apply tension to the first steering wire 130 and/or the second steering wire 132 to shift the elongate sheath 140 between the relaxed configuration, the deflected configuration, and the straightened configuration. In some embodiments, the first carriage member 160 and the second carriage member 170 may be configured to axially pass by each other as they axially translate along the threaded portion of the elongate shaft 122 and/or within the handle housing and/or the handle shell 112.

In some embodiments, actuation of the axial translation mechanism 120 may be configured to selectively apply tension to the first steering wire 130 and/or the second steering wire 132 to shift the elongate sheath 140 between the relaxed configuration, the deflected configuration, and the straightened configuration. In some embodiments, actuation of the axial translation mechanism 120 may be configured to selectively apply tension to the first steering wire 130 and/or the second steering wire 132 to bend and/or deflect the distal portion 144 and/or the first curve 146 of the elongate sheath 140. As discussed herein, the axial translation mechanism 120 may be configured to shift the elongate sheath 140 between the relaxed configuration, the deflected configuration wherein the distal portion 144 of the elongate sheath 140 is bent in the first direction from the relaxed configuration, and the straightened configuration wherein the distal portion 144 of the elongate sheath 140 is bent in the second direction from the relaxed configuration.

The first steering wire 130 may be configured to engage the axial translation mechanism 120 to bend and/or deflect the distal portion 144 and/or the first curve 146 of the elongate sheath 140 in the first direction toward the handle assembly 110 and/or the handle housing, toward and/or to the deflected configuration. The second steering wire 132 may be configured to engage the axial translation mechanism 120 to bend and/or deflect the distal portion 144 and/or the first curve 146 of the elongate sheath 140 in the second direction opposite the first direction and away from the handle assembly 110 and/or the handle housing, toward and/or to the straightened configuration. The tension applied by the axial translation mechanism 120 may be sufficient to overcome the self-bias of the elongate sheath 140 toward the normal or relaxed configuration and bend and/or deflect the distal portion 144 and/or the first curve 146 of the elongate sheath 140 in the first direction and/or the second direction.

In some embodiments, clockwise rotation of the elongate shaft 122 may be configured to and/or may cause proximal movement and/or proximal axial translation of the first carriage member 160. Proximal movement and/or proximal axial translation of the first carriage member 160 may apply tension to the first steering wire 130, thereby shifting the elongate sheath 140 toward and/or to the deflected configuration. In some embodiments, clockwise rotation of the elongate shaft 122 may be configured to and/or may cause distal movement and/or distal axial translation of the second carriage member 170. Distal movement and/or distal axial translation of the second carriage member 170 may release stored tension within the second steering wire 132 and/or may permit the second steering wire 132 to translate distally within the elongate sheath 140 such that the distal pull ring 150 may be pulled by the first steering wire 130.

In some embodiments, counterclockwise rotation of the elongate shaft 122 may be configured to and/or may cause proximal movement and/or proximal axial translation of the second carriage member 170. Proximal movement and/or proximal axial translation of the second carriage member 170 may apply tension to the second steering wire 132, thereby shifting the elongate sheath 140 toward and/or to the straightened configuration. In some embodiments, clockwise rotation of the elongate shaft 122 may be configured to and/or may cause distal movement and/or distal axial translation of the first carriage member 160. Distal movement and/or distal axial translation of the first carriage member 160 may release stored tension within the first steering wire 130 and/or may permit the first steering wire 130 to translate distally within the elongate sheath 140 such that the distal pull ring 150 may be pulled by the second steering wire 132.

Returning to FIGS. 1-2, in some embodiments, the handle assembly 110 and/or the handle shell 112 may comprise a non-visual torque indicator 180 configured to convey, provide, and/or communicate a rotational position of the handle assembly 110 and/or the handle shell 112 relative to a neutral position of the handle assembly 110 and/or the handle shell 112 to a user without the user needing to look at the handle assembly 110 and/or the handle shell 112 (e.g., without the user needing to look away from a fluoroscopy screen, for example). A non-visual torque indicator (such as the non-visual torque indicator 180 described herein) on the handle (e.g., on the handle assembly 110 and/or the handle shell 112) may improve the user interface during a procedure with a steerable catheter (such as the steerable catheter 100 described herein). In some procedures, a steerable catheter must be able to be torqued and/or deflected using only one hand. In some embodiments, the non-visual torque indicator may improve control over the steerable catheter during the procedure. In some embodiments, the non-visual torque indicator may improve ease of use and/or intuitiveness of the steerable catheter. Handle design is a differentiating factor that may influence a user's opinion on a product. The disclosure describes one or more handle designs intended to address one or more of the above factors and/or characteristics.

In some embodiments, the non-visual torque indicator 180 may comprise a tactile feature formed on a hand grip portion 111 of the handle housing and/or the handle shell 112. As such, the non-visual torque indicator 180 and/or the tactile feature may be felt by the user's hand as the user grips the hand grip portion 111 of the handle housing and/or the handle shell 112, thereby negating a need to look at the handle assembly 110, the handle housing, and/or the handle shell 112 to determine an orientation and/or the rotational position thereof relative to the neutral position. In some embodiments, the non-visual torque indicator 180 and/or the tactile feature may comprise a bumped-out region 182 of the handle housing and/or the handle shell 112 extending longitudinally along the handle housing and/or the handle shell 112. In some embodiments, the bumped-out region 182 of the handle housing and/or the handle shell 112 may be disposed between a proximal end of the hand grip portion 111 of the handle housing and/or the handle shell 112 and a distal end of the hand grip portion 111 of the handle housing and/or the handle shell 112. In some embodiments, the non-visual torque indicator 180 and/or the tactile feature may extend along more than 50% of a length 184 of the hand grip portion 111 of the handle housing and/or the handle shell 112. In some embodiments, the non-visual torque indicator 180 and/or the tactile feature may extend along less than 100% of the length 184 of the hand grip portion 111 of the handle housing and/or the handle shell 112. In some alternative configurations, the non-visual torque indicator 180 and/or the tactile feature may extend along 100% of the length 184 (e.g., an entire length) of the hand grip portion 111 of the handle housing and/or the handle shell 112. Other configurations are also contemplated.

In at least some embodiments, the handle assembly 110 may be adapted and/or configured to permit and/or to facilitate ambidextrous operation of the steerable catheter 100, the steering mechanism, and/or the knob 118. For example, in FIG. 1, the user could be positioned facing the handle assembly 110 from a front side (e.g., facing into the page and positioned in front of the handle assembly 110 as shown) or from a back side (e.g., facing out from the page and positioned behind the handle assembly 110 as shown). In some embodiments, the hand grip portion 111 of the handle housing and/or the handle shell 112 may be configured to receive and/or be gripped by a user's left hand or a user's right hand, depending on which side of the handle assembly 110 the user is positioned on.

In some embodiments, the non-visual torque indicator 180, the tactile feature, and/or the bumped-out region 182 may be curved, as seen in the example configuration of FIG. 1. In some embodiments, the non-visual torque indicator 180, the tactile feature, and/or the bumped-out region 182 may be configured to correspond to a user's natural grip and/or hand shape. In some embodiments, the non-visual torque indicator 180, the tactile feature, and/or the bumped-out region 182 may form a longitudinal rib, as seen in the example configurations of FIGS. 4 and 5. In some embodiments, the longitudinal rib may be oriented substantially parallel to the central longitudinal axis of the handle assembly 110. In some embodiments, the longitudinal rib may have substantially parallel sides extending outward from the handle housing and/or the handle shell 112.

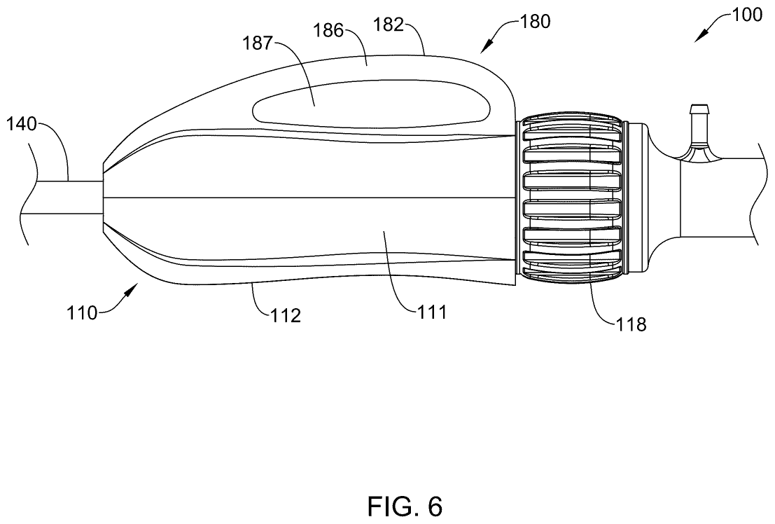

As shown in FIG. 6, in some embodiments, the non-visual torque indicator 180, the tactile feature, and/or the bumped-out region 182 may comprise a handle 186 at least partially defining an aperture 187 configured to receive a user's left fingers as the user's left hand grips the hand grip portion 111 of the handle housing and/or the handle shell 112 or a user's right fingers as the user's right hand grips the hand grip portion 111 of the handle housing and/or the handle shell 112. In some embodiments, a medial portion of the handle 186 may be spaced away from the hand grip portion 111 of the handle housing and/or the handle shell 112 and opposing ends of the handle 186 may be fixedly attached to and/or monolithically formed with the hand grip portion 111 of the handle housing and/or the handle shell 112. In at least some embodiments, the aperture 187 may be at least partially defined by the handle 186 and at least partially defined by the hand grip portion 111 of the handle housing and/or the handle shell 112. The user's left fingers or the user's right fingers may be extended through the aperture 187 when the user grips the hand grip portion 111. In some embodiments, the handle 186 may resemble and/or may be similar to a mug handle. Other configurations are also contemplated.

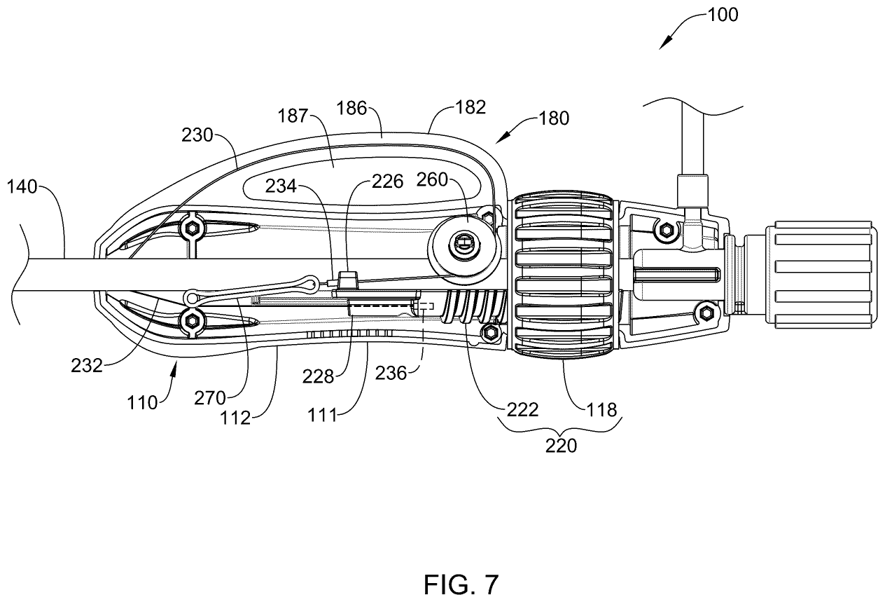

In FIG. 7, selected aspects of an alternative configuration of the steerable catheter 100 of FIG. 6 are illustrated, wherein some internal and/or functional elements of the steerable catheter 100 have been changed. In some embodiments, various disclosed elements of the configurations may be used in combination. In FIG. 7, a portion of the handle housing and/or the handle shell 112 has been removed to show some internal components of the handle assembly 110.

In some embodiments, the handle assembly 110 may comprise an axial translation mechanism 220. In some embodiments, the axial translation mechanism 220 may include a threaded member 222 slidably disposed within the handle housing and/or the handle shell 112. In some embodiments, the axial translation mechanism 220 may include the knob 118. In some embodiments, the knob 118 may be disposed around and/or may be configured to rotate around, and/or relative to, at least a portion of the handle housing and/or the handle shell 112. In some embodiments, the knob 118 may be configured to engage the threaded member 222 such that rotation of the knob 118 relative to the handle housing and/or the handle shell 112 causes axial translation of the threaded member 222 proximally and/or distally within the handle housing and/or the handle shell 112. In some embodiments, rotation of the knob 118 in a clockwise direction relative to the handle housing and/or the handle shell 112, as viewed along the steerable catheter 100 proximally to distally, may cause axial translation of the threaded member 222 distally within the handle housing and/or the handle shell 112. In some embodiments, rotation of the knob 118 in a counterclockwise direction relative to the handle housing and/or the handle shell 112, as viewed along the steerable catheter 100 proximally to distally, may cause axial translation of the threaded member 222 proximally within the handle housing and/or the handle shell 112. In some embodiments, the reverse and/or opposite configuration may be used, wherein clockwise rotation of the knob 118 moves the threaded member 222 proximally and counterclockwise rotation of the knob 118 moves the threaded member 222 distally. The orientation of the internal and external threads on the knob 118 and the threaded member 222, respectively, determine which direction of rotation is tied to which direction of axial translation. Some suitable but non-limiting materials for the axial translation mechanism 220, the threaded member 222, and/or the knob 118 are described below.

A first steering wire 230 may extend within the elongate sheath 140 from the handle housing and/or the handle shell 112 to the distal pull ring 150 (e.g., FIG. 3) in a manner similar to and/or consistent with the first steering wire 130. A second steering wire 232 may extend within the elongate sheath 140 from the handle housing and/or the handle shell 112 to the distal pull ring 150 (e.g., FIG. 3) in a manner similar to and/or consistent with the second steering wire 132. The second steering wire 232 may be disposed on an opposite side of the elongate sheath 140 from the first steering wire 230 relative to a central longitudinal axis of the elongate sheath 140. Tension may be applied to the first steering wire 230 and/or the second steering wire 232 as described herein to bend and/or deflect the distal portion 144 and/or the first curve 146 of the elongate sheath 140 (e.g., FIG. 1). The first steering wire 230 may be configured to engage the axial translation mechanism 220 and/or the threaded member 222 to bend and/or deflect the distal portion 144 and/or the first curve 146 of the elongate sheath 140 in the first direction toward the handle housing and/or the handle shell 112, toward and/or to the deflected configuration (e.g., FIG. 1). The second steering wire 232 may be configured to engage the axial translation mechanism 220 and/or the threaded member 222 to bend and/or deflect the distal portion 144 and/or the first curve 146 of the elongate sheath 140 in the second direction opposite the first direction and away from the handle housing and/or the handle shell 112, toward and/or to the straightened configuration (e.g., FIG. 1).

In some embodiments, the steerable catheter 100 may include a pulley wheel 260 disposed within the handle housing and/or the handle shell 112. The pulley wheel 260 may be engaged with the first steering wire 230 via a circumferential channel extending around the pulley wheel 260. In some embodiments, the steerable catheter 100 may include a tensioning member 270. The tensioning member 270 may couple a first end (e.g., a proximal end) of the first steering wire 230 to the handle housing and/or the handle shell 112. In at least some embodiments, the proximal end of the first steering wire 230 may be fixedly coupled to the handle housing and/or the handle shell 112 by the tensioning member 270. In some embodiments, the pulley wheel 260 may engage the first steering wire 230 at a position proximal of the tensioning member 270. In some embodiments, the tensioning member 270 may be coupled to the handle housing and/or the handle shell 112 at a position distal of the proximal end of the first steering wire 230. In some embodiments, the tensioning member 270 may be an elastic polymer, as shown in FIG. 7. In another example, the tensioning member 270 may be a coil spring (not shown). Other configurations are also contemplated. The tensioning member 270 may be configured to apply a small, non-biasing amount of tension to the first steering wire 230 when the distal portion 144 and/or the first curve 146 of the elongate sheath 140 is disposed in the normal or relaxed configuration and/or when the distal portion 144 and/or the first curve 146 of the elongate sheath 140 is bent and/or deflected in the second direction, toward and/or to the straightened configuration. The purpose of the tensioning member 270 is to prevent the first steering wire 230 from disengaging from the pulley wheel 260 when there is no tension being applied to the first steering wire 230 by the axial translation mechanism 220 and/or the threaded member 222 (e.g., in the normal or relaxed configuration, or toward and/or in the straightened configuration) by tightly holding the first steering wire 230 around the pulley wheel 260. Some suitable but non-limiting materials for the pulley wheel 260 and/or the tensioning member 270 are described below.

In some embodiments, the steerable catheter 100 may include one or more ribs, projections, bosses, or posts extending transversely within the handle housing and/or the handle shell 112 between opposing walls and/or opposite sides of the handle housing and/or the handle shell 112 in addition to the pulley wheel 260. In some embodiments, the one or more ribs, projections, bosses, or posts may extend completely across an interior of the handle housing and/or the handle shell 112 from one side of the handle housing and/or the handle shell 112 to an opposing side of the handle housing and/or the handle shell 112. In some embodiments, the first steering wire 230 may be routed around and/or may slide past the one or more ribs, projections, bosses, or posts to guide the first steering wire 230 between the pulley wheel 260 and the elongate sheath 140.

The threaded member 222 may include a first catch 226 extending transversely from the threaded member 222 in a first lateral direction. The first steering wire 230 may extend and/or pass through the first catch 226. The first steering wire 230 may include a first stop element 234 configured to engage with the axial translation mechanism 220 and/or the first catch 226 of the threaded member 222 when the threaded member 222 slides in a distal direction within the handle housing and/or the handle shell 112 to apply tension to the first steering wire 230. The tension applied by the axial translation mechanism 220 and/or the threaded member 222 may be sufficient to overcome the self-bias of the elongate sheath 140 toward the normal or relaxed configuration and bend and/or deflect the distal portion 144 and/or the first curve 146 of the elongate sheath 140 in the first direction.

The threaded member 222 may include a second catch 228 extending transversely from the threaded member 222 in a second lateral direction opposite the first lateral direction. The second steering wire 232 may extend and/or pass through the second catch 228. The second steering wire 232 may include a second stop element 236 configured to engage with the axial translation mechanism 220 and/or the second catch 228 of the threaded member 222 when the threaded member 222 slides in a proximal direction within the handle housing and/or the handle shell 112 to apply tension to the second steering wire 232. The tension applied by the axial translation mechanism 220 and/or the threaded member 222 may be sufficient to overcome the self-bias of the elongate sheath 140 toward the normal or relaxed configuration and bend and/or deflect the distal portion 144 and/or the first curve 146 of the elongate sheath 140 in the second direction.

The pulley wheel 260 permits the threaded member 222 to apply tension to both the first steering wire 230 and the second steering wire 232, depending upon which direction the threaded member 222 is moving. Tension applied to the first steering wire 230 and the second steering wire 232 causes bending and/or deflection of the distal portion 144 and/or the first curve 146 of the elongate sheath 140 away from the normal or relaxed configuration. Since both steering wires extend proximally from the distal pull ring 150, the pulley wheel 260 is needed to reverse the direction of the first steering wire 230 relative to the second steering wire 232 within the handle housing and/or the handle shell 112 such that the threaded member 222 is able to selectively apply tension to both the first steering wire 230 and the second steering wire 232 by moving in opposite directions. In one or more alternative configurations, the handle housing and/or the handle shell 112 may include an internal rib, an internal protrusion, or other features disposed therein, in place of the pulley wheel 260, around which the first steering wire 230 may extend and reverse direction to function as described herein.

In some embodiments, the first steering wire 230 may extend from the pulley wheel 260 distally to the elongate sheath 140 without engaging any additional portions or elements of the handle housing and/or the handle shell 112 (such as the one or more ribs, projections, bosses, or posts). In some embodiments, the first steering wire 230 may extend from the pulley wheel 260 through the handle 186 (e.g., through the medial portion of the handle 186) to and/or into the elongate sheath 140 at a location proximate a distal end of the handle housing and/or the handle shell 112, as seen in FIG. 7. In some embodiments, routing the first steering wire 230 through the handle 186 may permit a larger bend radius to be formed in the first steering wire 230 than if the first steering wire 230 routed more directly from the pulley wheel 260 to the elongate sheath 140, thereby reducing fatigue in the first steering wire 230. While not expressly illustrated, the handle 186 may include one or more ribs, projections, bosses, or posts to serve as guides for the first steering wire 230 extending therethrough.

When the threaded member 222 is disposed in a central position, the distal portion 144 and/or the first curve 146 of the elongate sheath 140 may be disposed in the normal or relaxed configuration. When the threaded member 222 is disposed in the central position, substantially no tension is being applied to the first steering wire 230 and/or the second steering wire 232. As the threaded member 222 is axially translated proximally and/or distally within the handle housing and/or the handle shell 112, the threaded member 222 of the axial translation mechanism 220 may engage with the first steering wire 230 and/or the second steering wire 232 to apply tension thereto to bend and/or deflect the distal portion 144 and/or the first curve 146 of the elongate sheath 140 as described herein. Additionally, when the threaded member 222 is disposed in the central position, the first catch 226 may be engaged with the first stop element 234 but tension is not being applied to the first steering wire 230, and the second catch 228 may be engaged with the second stop element 236 but tension is not being applied to the second steering wire 232. As such, the central position of the threaded member 222 may be tension-neutral with respect to the first steering wire 230 and the second steering wire 232.

When the threaded member 222 is moved from the central position toward and/or until disposed in a proximal position, tension may be applied to the second steering wire 232 and the distal portion 144 and/or the first curve 146 of the elongate sheath 140 may be bent and/or deflected in the second direction away from the handle housing and/or the handle shell 112, or toward and/or to the straightened configuration. In moving the threaded member 222 proximally within the handle housing and/or the handle shell 112 from the central position, the second catch 228 engages the second stop element 236 and thereafter translates the second stop element 236 proximally, thereby applying tension to the second steering wire 232. The first stop element 234 may disengage from the axial translation mechanism 220, the threaded member 222, and/or the first catch 226 to release tension on the first steering wire 230 when the threaded member 222 slides in the proximal direction within the handle housing and/or the handle shell 112. Accordingly, when the threaded member 222 is moved proximally from the central position, the first catch 226 may be disengaged from the first stop element 234 and the first catch 226 may slide proximally along and/or over the first steering wire 230. The first stop element 234 may be configured to float relative to (e.g., the first stop element 234 may not be directly fixed to) the axial translation mechanism 220, the threaded member 222, and/or the first catch 226 when the threaded member 222 slides in the proximal direction within the handle housing and/or the handle shell 112. As such, slack would form in the first steering wire 230, which would allow the first steering wire 230 to disengage from the pulley wheel 260, except for the tension applied by the tensioning member 270. The tensioning member 270 holds the first steering wire 230 tightly around the pulley wheel 260 while no tension is being applied to the first steering wire 230 by the threaded member 222 and/or the first catch 226. The tensioning member 270 absorbs any slack that would be formed in the first steering wire 230 due to the first catch 226 being disengaged from the first stop element 234 and prevents the first steering wire 230 from disengaging from the pulley wheel 260.

When the threaded member 222 is moved from the central position toward and/or until disposed in a distal position, tension may be applied to the first steering wire 230 and the distal portion 144 and/or the first curve 146 of the elongate sheath 140 may be bent and/or deflected in the first direction toward the handle housing and/or the handle shell 112, or toward and/or to the deflected configuration. In moving the threaded member 222 distally within the handle housing and/or the handle shell 112 from the central position, the first catch 226 engages the first stop element 234 and thereafter translates the first stop element 234 distally, thereby applying tension to the first steering wire 230. The second stop element 236 may disengage from the axial translation mechanism 220, the threaded member 222, and/or the second catch 228 to release tension on the second steering wire 232 when the threaded member 222 slides in the distal direction within the handle housing and/or the handle shell 112. Accordingly, when the threaded member 222 is moved distally from the central position, the second catch 228 may be disengaged from the second stop element 236 and the second catch 228 may slide distally along and/or over the second steering wire 232. The second stop element 236 may be configured to float relative to (e.g., the second stop element 236 may not be directly fixed to) the axial translation mechanism 220, the threaded member 222, and/or the second catch 228 when the threaded member 222 slides in the distal direction within the handle housing and/or the handle shell 112. As such, slack forms in the second steering wire 232 due to the second catch 228 being disengaged from the second stop element 236. As the threaded member 222 is translated distally from the proximal position and/or the central position, the first catch 226 engages the first stop element 234 and the first steering wire 230 is thereafter pulled around the pulley wheel 260 and tension applied by the tensioning member 270 is relieved as tension is instead applied to the first steering wire 230 by the first catch 226 and/or the threaded member 222.

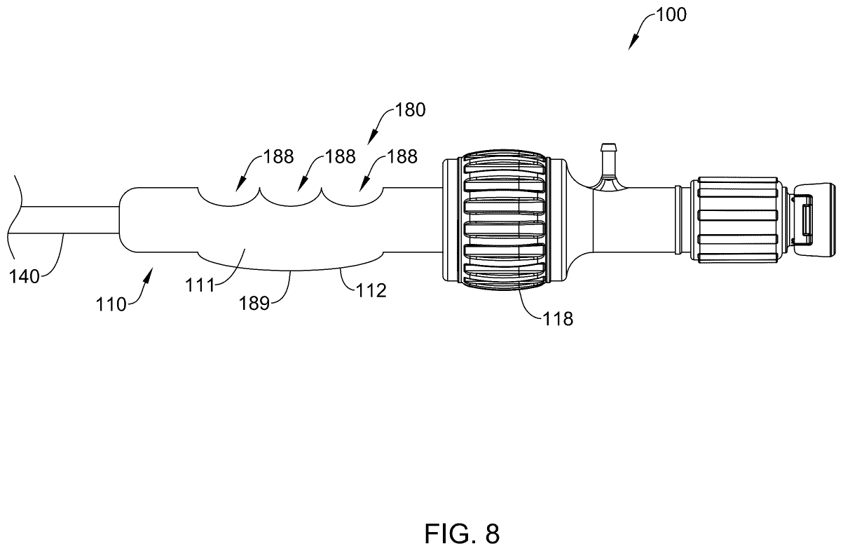

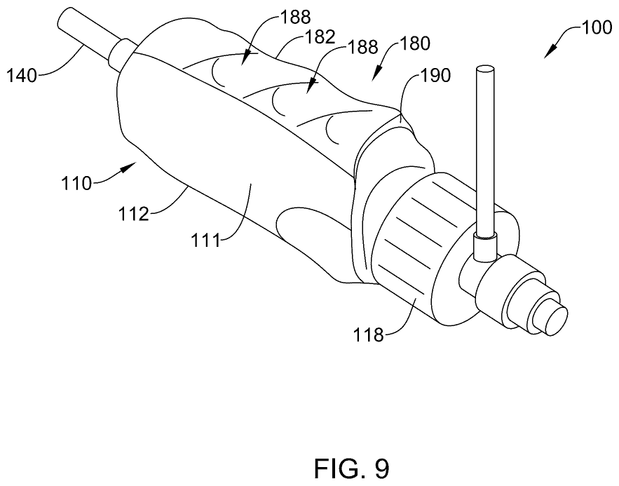

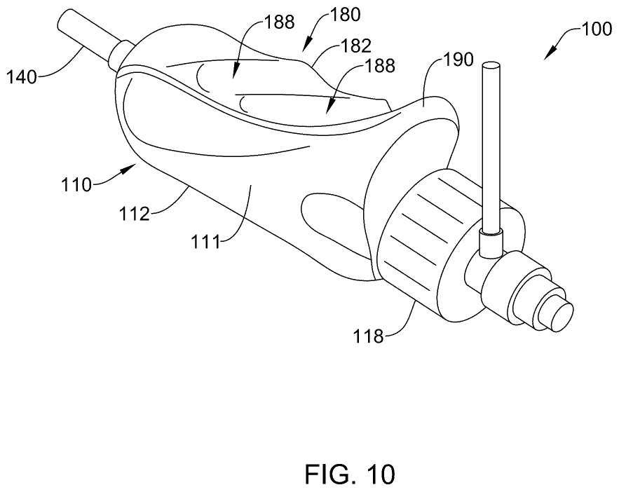

Turning now to FIGS. 8-10, several different form factors for the handle assembly 110, the hand grip portion 111, the non-visual torque indicator 180, and/or the tactile feature are illustrated. In some embodiments, the hand grip portion 111 of the handle housing and/or the handle shell 112 and/or the non-visual torque indicator 180 may comprise a plurality of grooves 188 configured to receive a user's fingers therein. For example, the plurality of grooves 188 may be configured to receive a user's left fingers as the user's left hand grips the hand grip portion 111 of the handle housing and/or the handle shell 112 or a user's right fingers as the user's right hand grips the hand grip portion 111 of the handle housing and/or the handle shell 112. In some embodiments, the hand grip portion 111, the non-visual torque indicator 180, and/or the tactile feature may comprise the plurality of grooves 188 positioned opposite a bumped-out region 189, as seen in FIG. 8. In some embodiments, the hand grip portion 111, the non-visual torque indicator 180, and/or the tactile feature may comprise the plurality of grooves 188 positioned within the bumped-out region 182, as seen in FIGS. 9 and 10. In some embodiments, the hand grip portion 111 and/or the non-visual torque indicator 180 may comprise a proximal end portion 190 that extends radially outward from central longitudinal axis of the handle assembly 110 further than any other feature of the hand grip portion 111 and/or the non-visual torque indicator 180. In some embodiments, the proximal end portion 190 may form a proximal stop configured to prevent the user's hand from moving and/or sliding onto the knob 118. Other configurations, including combinations thereof, are also contemplated.

The materials that can be used for the various components of the steerable catheter (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the handle assembly, the elongate sheath, the steering mechanism, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C- 22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof, or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. A steerable catheter, comprising:
a handle assembly comprising a handle shell and a steering mechanism; and
an elongate sheath extending distally from the handle assembly;
wherein the steering mechanism includes an axial translation mechanism coupled to a first steering wire extending within the elongate sheath and a second steering wire extending within the elongate sheath;
wherein the first steering wire is configured to cooperate with the axial translation mechanism to bend a distal portion of the elongate sheath in a first direction;
wherein the second steering wire is configured to cooperate with the axial translation mechanism to bend the distal portion of the elongate sheath in a second direction opposite the first direction;
wherein the handle assembly comprises a non-visual torque indicator configured to convey a rotational position of the handle assembly relative to a neutral position.

2. The steerable catheter of claim 1, wherein the non-visual torque indicator comprises a tactile feature formed on a hand grip portion of the handle shell.

3. The steerable catheter of claim 2, wherein the tactile feature comprises a bumped-out region of the handle shell extending longitudinally along the handle shell.

4. The steerable catheter of claim 3, wherein the bumped-out region is disposed between a proximal end of the hand grip portion and a distal end of the hand grip portion.

5. The steerable catheter of claim 2, wherein the tactile feature extends along more than 50% of a length of the hand grip portion.

6. The steerable catheter of claim 5, wherein the tactile feature extends along less than 100% of the length of the hand grip portion.

7. The steerable catheter of claim 2, wherein the hand grip portion comprises a plurality of grooves configured to receive a user's fingers therein.

8. The steerable catheter of claim 1, wherein the axial translation mechanism comprises:
an elongate shaft having a threaded portion disposed within the handle shell;
a first carriage member operatively engaged with the threaded portion; and
a second carriage member operatively engaged with the threaded portion;
wherein rotation of the elongate shaft is configured to translate the first carriage member and the second carriage member in opposite directions along the elongate shaft.

9. The steerable catheter of claim 8, wherein the handle assembly comprises a knob fixedly attached to the elongate shaft outside of the handle shell, wherein the knob is rotatable relative to the handle shell to rotate the threaded portion within the handle shell.

10. The steerable catheter of claim 9, wherein the knob is axially offset from the handle shell.

11. A steerable catheter, comprising:
a handle assembly comprising a handle shell and a steering mechanism; and
an elongate sheath extending distally from the handle assembly;
wherein the steering mechanism includes an axial translation mechanism;
wherein a first steering wire extends distally from the axial translation mechanism to a distal pull ring disposed within the elongate sheath;
wherein a second steering wire extends distally from the axial translation mechanism to the distal pull ring, the second steering wire being disposed on an opposite side of the elongate sheath from the first steering wire relative to a central longitudinal axis of the elongate sheath;
wherein the first steering wire is configured to cooperate with the axial translation mechanism to bend a distal portion of the elongate sheath in a first direction;
wherein the second steering wire is configured to cooperate with the axial translation mechanism to bend the distal portion of the elongate sheath in a second direction opposite the first direction;
wherein the handle assembly comprises a non-visual torque indicator configured to convey a rotational position of the handle assembly relative to a neutral position.

12. The steerable catheter of claim 11, wherein the axial translation mechanism is configured to shift the elongate sheath between a relaxed configuration, a deflected configuration wherein the distal portion of the elongate sheath is bent in the first direction from the relaxed configuration, and a straightened configuration wherein the distal portion of the elongate sheath is bent in the second direction from the relaxed configuration.

13. The steerable catheter of claim 12, wherein actuation of the axial translation mechanism is configured to selectively apply tension to the first steering wire and the second steering wire to shift the elongate sheath between the relaxed configuration, the deflected configuration, and the straightened configuration.

14. The steerable catheter of claim 11, wherein the axial translation mechanism comprises:
an elongate shaft having a threaded portion disposed within the handle shell;
a first carriage member operatively engaged with the threaded portion; and
a second carriage member operatively engaged with the threaded portion;
wherein rotation of the elongate shaft is configured to translate the first carriage member and the second carriage member in opposite directions along the elongate shaft.

15. The steerable catheter of claim 14, wherein the handle assembly comprises a knob fixedly attached to the elongate shaft outside of the handle shell, wherein the knob is rotatable relative to the handle shell to rotate the threaded portion within the handle shell.

16. The steerable catheter of claim 15, wherein the knob is proximally offset from the handle shell.

17. The steerable catheter of claim 15, wherein the knob is distally offset from the handle shell.

18. The steerable catheter of claim 14, wherein the threaded portion comprises a first helical thread extending in a first helical direction overlapping a second helical thread extending in a second helical direction.

19. The steerable catheter of claim 14, wherein the first carriage member is coupled to the first steering wire and the second carriage member is coupled to the second steering wire.

20. A steerable catheter, comprising:
a handle assembly comprising a handle shell and a steering mechanism; and
an elongate sheath extending distally from the handle assembly;
wherein the steering mechanism includes an axial translation mechanism;
wherein a first steering wire extends distally from the axial translation mechanism to a distal pull ring disposed within the elongate sheath;
wherein a second steering wire extends distally from the axial translation mechanism to the distal pull ring, the second steering wire being disposed on an opposite side of the elongate sheath from the first steering wire relative to a central longitudinal axis of the elongate sheath;
wherein the first steering wire is configured to cooperate with the axial translation mechanism to bend a distal portion of the elongate sheath in a first direction;
wherein the second steering wire is configured to cooperate with the axial translation mechanism to bend the distal portion of the elongate sheath in a second direction opposite the first direction;
wherein the handle assembly comprises a non-visual torque indicator configured to convey a rotational position of the handle assembly relative to a neutral position;
wherein the handle assembly is configured to permit ambidextrous operation of the steering mechanism.
